(19) United States
(12) Patent Application Publication
(10) Pub. No.: US 2010/0144800 A1
(43) Pub. Date: Jun. 10, 2010

(54) EXTENDED RELEASE TABLET FORMULATION OF NIACIN

(75) Inventors: Chayapathy Issa, Madanpalle (IN); Praveen Raheja, New Delhi (IN); Sachin Arora, Muzaffarnagar (IN); Rajesh Gandhi, Gurgaon (IN); Rajeev Shanker Mathur, Gurgaon (IN)

Correspondence Address: Ranbaxy Inc., Intellectual Property Department, 600 College Road East, PRINCETON, NJ, 08540, US

(73) Assignee: RANBAXY LABORATORIES LIMITED, Gurgaon (IN)

(21) Appl. No.: 12/482,290

(22) Filed: Jun. 10, 2009

(30) Foreign Application Priority Data
Jun. 11, 2008 (IN) 1386/DEL/2008

(51) Int. Cl. A61K 31/455; A61P 9/10

(52) U.S. Cl. 514/356

(57) ABSTRACT

The present invention relates to an extended release tablet formulation comprising niacin and a release retarding agent selected from hydroxypropyl cellulose, polyethylene oxide or mixtures thereof.

### FIELD OF THE INVENTION

The present invention relates to an extended release tablet formulation comprising niacin and a release retarding agent selected from hydroxypropyl cellulose, polyethylene oxide or mixtures thereof and process of preparation of such formulations.

### BACKGROUND OF THE INVENTION

Niacin is also known as nicotinic acid. It is chemically, 3-pyridine carboxylic acid, and has been used for many years in the treatment of hyperlipidemia and hypercholesterolemia. This compound has been known to exhibit the beneficial effects of reducing total cholesterol, very low density lipoprotein (VLDL-Cholesterol) and VLDL-cholesterol remnants, LDL-cholesterol, triglycerides and apolipoprotein, while increasing desirable HDL-cholesterol.

A fast release nicotinic acid has conventionally been administered three times per day after meals, but cutaneous flushing often occurs in the hyperlipidemics to whom the nicotinic acid is administered.

In order to avoid or alleviate the cutaneous flushing resulting from nicotinic acid therapy, a number of agents have been suggested. Another method of avoiding or reducing the side effects associated with fast release niacin is the use of extended or sustained release formulations. Extended or sustained release formulations are designed to slowly release the active ingredient from the tablet or capsule, which allows a reduction in dosing frequency as compared to the typical dosing frequency associated with conventional or fast dosage forms. The slow drug release reduces and prolongs blood levels of the drug and, thus, minimizes or lessens the cutaneous flushing side effects that are associated with conventional or fast release niacin products.

The extended release tablet formulations of niacin are known in the art.

U.S. Pat. No. 5,268,181, assigned to Upsher-Smith Laboratories Inc., discloses a therapeutic method to treat hyperlipidemia by administering to a human patient a single daily dose of a prolonged release dosage form of niacin, so that nocturnal cholesterol synthesis is effectively suppressed. Also, disclosed is a sustained or controlled release tablet comprising: a water soluble medicament, a hydroxypropyl methylcellulose having sustaining action, a pharmaceutical binding agent, and a hydrophobic component.

U.S. Pat. No. 6,080,428, assigned to Kos Pharmaceuticals, discloses an orally administered antihyperlipidemia composition comprising from about 250 to about 3000 parts by weight of nicotinic acid, and from about 5 to about 50 parts by weight of hydroxypropyl methylcellulose.

WO2007/120385, assigned to Kos Pharmaceuticals, discloses an extended-release matrix formulation capable of being directly compressed into tablets comprising niacin, a release-retarding agent, and other excipients. Hydroxypropyl methylcellulose having a methoxyl degree of substitution of about 1.39 to about 1.41 and a hydroxypropoxyl molar substitution of about 0.20 to about 0.22 is disclosed as the preferable polymer.

The extended release tablet formulation of the present invention provides alternate formulations that may be bioequivalent to formulations of NIASPAN® (approved by the U.S. Food and Drug Administration containing niacin extended release commercially available in 500, 750, and 1000mg strengths).

### SUMMARY OF THE INVENTION

According to one embodiment there is provided an extended release tablet formulation comprising niacin and a release retarding agent selected from hydroxypropyl cellulose, polyethylene oxide or mixtures thereof.

According to another embodiment there is provided an extended release tablet formulation comprising niacin and 5-50% w/w of a release retarding agent selected from hydroxypropyl cellulose, polyethylene oxide or mixtures thereof.

According to still another embodiment there is provided an extended release tablet formulation comprising niacin, 5-25% w/w of hydroxypropyl cellulose and one or more pharmaceutically acceptable excipients.

According to another embodiment there is provided an extended release tablet formulation comprising niacin, 15-35% w/w of polyethylene oxide and one or more pharmaceutically acceptable excipients.

According to another embodiment there is provided an extended release tablet formulation comprising niacin, 5-15% w/w of hydroxypropyl cellulose, 5-25% w/w of polyethylene oxide and one or more pharmaceutically acceptable excipients.

According to still another embodiment there is provided a process for preparing the extended release tablet formulation disclosed in the various embodiments of the specification.

### DETAILED DESCRIPTION OF THE INVENTION

According to one embodiment, the extended release tablet formulation comprises niacin and release retarding agent. The formulation may further comprise one or more pharmaceutically acceptable excipients.

The term ‘Niacin’ refers to nicotinic acid compound or any mixtures thereof, and specifically includes, but are not limited to the following: nicotinic acid, nicotinyl alcohol tartrate, D-glucitol hexanicotinate, aluminum nicotinate, niceritrol, D-L-alpha-tocopheryl nicotinate, 6-OH-nicotinic acid, nicotinuaria acid, nicotinamide, nicotinamide-N-oxide, 6-OH-nicotinamide, NAD, N-methyl-2-pyridine-8-carboxamide, N-methyl-nicotinamide, N-ribosyl-2-pyridone-5-carboxide, N-methyl-4-pyridone-5-carboxamide, bradilian, sorbinicate, hexanicite, ronitol, and esters of nicotinic acid such as lower alcohol esters like methyl, ethyl, propyl or butyl esters. Each of such derivatives or compounds will be collectively referred to hereinafter by ‘Niacin’. The amount of niacin may range from about 250 mg to about 3000 mg, for example—from about 500 mg to about 2500 mg. Niacin may be daily dosed in increments of, for example, 250 mg, 500 mg, 750 mg, 1000 mg, 1500 mg, 2000 mg, 2500 mg and 3000 mg. The tablets of the present invention may include niacin in dosage amounts of, for example—250 mg, 375 mg, 500 mg, 750 mg and 1000 mg.

The ‘release retarding agent’ is selected from hydroxypropyl cellulose, polyethylene oxide or mixtures thereof. The specific grades of hydroxypropyl cellulose e.g. HPC-H, HPC-M, HPC-HX, HPC-HXF by Hercules Inc. and polyethylene oxide e.g. PEO-27, PEO-18, PEO-15, PEO-8, PEO-4 by Sumitomo Seika Chemicals Co., Polyox WSR 1105, Polyox WSR 303 by Dow Chemicals., providing desired release profile may be used. The release retarding agent is present in an amount of 5-50% by weight of the total formulation. For example, 5-25% w/w of hydroxypropyl cellulose or 15-35% w/w of polyethylene oxide or mixture of 5-15% w/w of hydroxypropyl cellulose and 5-25% w/w of polyethylene oxide.

The ‘pharmaceutically acceptable excipients’ may be selected from one or more of diluents, binders, disintegrants, lubricants, glidants and coloring agents.

The ‘diluent’ may be selected from one or more of saccharides such as lactose, sucrose and glucose; sugar alcohols such as mannitol, sorbitol, xylitol and maltitol.

The ‘binder’ may be selected from one or more of starches such as starch, pregelatinized starch and modified starch; cellulose derivatives such as hydroxypropyl methylcellulose, hydroxypropyl cellulose and methylcellulose; gums such as xanthan gum, gum acacia and tragacanth; and water soluble vinylpyrrolidone polymers such as polyvinyl pyrrolidone, copolymer of vinyl pyrrolidone and vinyl acetate.

The ‘disintegrant’ may be selected from one or more of crospovidone, hydroxypropyl cellulose, pregelatinized starch, sodium starch glycolate and croscarmellose sodium.

The ‘lubricant’ may be selected from one or more of talc, stearic acid, colloidal silicon dioxide, magnesium stearate and sodium stearyl fumarate.

The extended release tablet formulation of the invention may comprise a non-functional coating.

The ‘non-functional coating’ may comprise polymers like hydroxypropyl cellulose, hydroxyethyl cellulose, hydroxypropyl methylcellulose, polyvinyl pyrrolidone, copolymer of vinyl pyrrolidone and vinyl acetate; plasticizers like polyethylene glycol, triacetin, dibutyl sebecate and diethyl tartrate; opacifying agents like titanium dioxide and talc; and coloring agents. Examples of such non-functional coats are commercially available Opadry® compositions.

According to another embodiment, the extended release tablet formulation of niacin may further comprise an HMG-CoA reductase inhibitor. HMG-CoA reductase inhibitors have also been used for many years to treat hyperlipidemia. Hence it would be advantageous to administer HMG-CoA reductase inhibitor along with niacin to achieve a balanced lipid alteration. The ‘HMG-CoA reductase inhibitors’ include, but are not limited to simvastatin, lovastatin, pravastatin free bases or pharmaceutically acceptable salts, solvates or mixtures thereof.

The extended release tablet formulation of the invention may be prepared by wet granulation, dry granulation or direct compression process. The wet granulation process involves use of water or any other suitable solvent. The dry granulation may involve use of roller compacter or any suitable technique.

According to one embodiment of the specification is provided a process for the preparation of an extended release tablet formulation, the process comprising the steps of:
a) preparing granules comprising niacin, a release retarding agent selected from hydroxypropyl cellulose, polyethylene oxide or mixtures thereof, and one or more pharmaceutically acceptable excipients;
b) blending the granules of step (a) with a release retarding agent selected from hydroxypropyl cellulose, polyethylene oxide or mixtures thereof, and optionally one or more pharmaceutically acceptable excipients; and,
c) compressing the blend of step (b) into a tablet.

The following non-limiting examples further illustrate the extended release tablet formulation of Niacin and process of making such formulation:

### EXAMPLE 1

| | 500 mg | 750 mg | 1000 mg |
|---|---|---|---|
| Ingredient | mg/tab | mg/tab | mg/tab |
| Intragranular | | | |
| Niacin | 500 | 750 | 1000 |
| Hydroxypropyl cellulose | 50 | 75 | 100 |
| (HPC-H) | | | |
| Polyvinylpyrrolidone | 17 | 25.5 | 34 |
| Purified water | q.s. | q.s. | q.s. |
| Extra granular | | | |
| Hydroxypropyl cellulose | 112 | 77.5 | 20 |
| (HPC-H) | | | |
| Stearic Acid | 7 | 10 | 12 |
| Core tablet weight | 686 | 938 | 1166 |
| Coating | | | |
| Opadry | 21 | 28 | 35 |

### EXAMPLE 2

| | 500 mg | 750 mg | 1000 mg |
|---|---|---|---|
| Ingredient | mg/tab | mg/tab | mg/tab |
| Intragranular | | | |
| Niacin | 500 | 750 | 1000 |
| Hydroxypropyl cellulose | 60 | 90 | 120 |
| (HPC-H) | | | |
| Polyethylene oxide | 30 | 45 | 60 |
| Polyvinylpyrrolidone | 17 | 25.5 | 34 |
| Purified water | q.s. | q.s. | q.s. |
| Extra granular | | | |
| Polyethylene oxide | 135 | 104.5 | 49 |
| Stearic Acid | 8 | 10.0 | 12 |
| Core tablet weight | 750 | 1025 | 1275 |
| Coating | | | |
| Opadry | 23 | 31 | 38 |

### EXAMPLE 3

| | 500 mg | 750 mg | 1000 mg |
|---|---|---|---|
| Ingredient | mg/tab | mg/tab | mg/tab |
| Intragranular | | | |
| Niacin | 500 | 750 | 1000 |
| Polyethylene oxide | 100 | 150 | 200 |
| Polyvinylpyrrolidone | 17 | 25.5 | 34 |
| Purified water | q.s. | q.s. | q.s. |
| Extra granular | | | |
| Polyethylene oxide | 150 | 122.5 | 70 |
| Stearic Acid | 8 | 10.0 | 12 |
| Core tablet weight | 775 | 1058 | 1316 |
| Coating | | | |
| Opadry | 23 | 32 | 40 |

### Brief Manufacturing Process:

1. Niacin along with hydroxypropyl cellulose and/or polyethylene oxide and polyvinylpyrollidone was sifted through BSS#30 sieve.
2. The material of step 1 was granulated in rapid mixer granulator using purified water.
3. The granules of step 2 were dried in fluid bed dryer at 50-60° C.
4. The dried granules of step 3 were sifted through BSS#25 sieve.
5. The extragranular hydroxypropyl cellulose or polyethylene oxide was sifted through BSS#30 sieve and was blended with the granules of step 4.
6. The extra granular stearic acid was sifted through BSS#30 sieve and was blended with the material of step 5.
7. The blend of step 6 was compressed to get tablets.
8. Opadry was dispersed in purified water.
9. The tablets of step 7 were coated using the dispersion of step 8 in a coating pan.

We claim:

1. An extended release tablet formulation comprising niacin, a release retarding agent selected from hydroxypropyl cellulose, polyethylene oxide or mixtures thereof, and one or more pharmaceutically acceptable excipients.

2. The tablet formulation of claim 1, wherein the tablet comprises 5-50% w/w of the release retarding agent.

3. The tablet formulation of claim 2, wherein the tablet comprises niacin, 5-25% w/w of hydroxypropyl cellulose and one or more pharmaceutically acceptable excipients.

4. The tablet formulation of claim 2, wherein the tablet comprises niacin, 15-35% w/w of polyethylene oxide and one or more pharmaceutically acceptable excipients.

5. The tablet formulation of claim 2, wherein the tablet comprises niacin, 5-15% w/w of hydroxypropyl cellulose, 5-25% w/w of polyethylene oxide and one or more pharmaceutically acceptable excipients.

6. The tablet formulation of claim 1, wherein the pharmaceutically acceptable excipients comprise one or more of diluents, binders, disintegrants, lubricants, glidants and coloring agents.

7. The tablet formulation of claim 1 further comprising an HMG-CoA reductase inhibitor.

8. The tablet formulation of claim 7, wherein the HMG-CoA reductase inhibitor is selected from one or more of simvastatin, lovastatin or pravastatin.

9. A process for the preparation of the extended release tablet formulation of claim 1, the process comprising the steps of:
a) preparing granules comprising niacin, a release retarding agent selected from hydroxypropyl cellulose, polyethylene oxide or mixtures thereof, and one or more pharmaceutically acceptable excipients;
b) blending the granules of step (a) with a release retarding agent selected from hydroxypropyl cellulose, polyethylene oxide or mixtures thereof, and optionally one or more pharmaceutically acceptable excipients; and,
c) compressing the blend of step (b) into a tablet.
